(19) United States
(12) Patent Application Publication
(10) Pub. No.: US 2012/0069418 A1
(43) Pub. Date: Mar. 22, 2012

(54) ELECTROCHROMIC FORMULATION, METHOD FOR THE PRODUCTION THEREOF, AND ORGANIC ELECTROCHROMIC COMPONENT

(76) Inventors: Andreas Kanitz, Hochstadt (DE); Marek Maleika, Furth (DE); Wolfgang Roth, Uttenreuth (DE)

(21) Appl. No.: 13/375,029

(22) PCT Filed: Apr. 20, 2010
(86) PCT No.: PCT/EP2010/055146

§ 371 Date: Nov. 29, 2011

(30) Foreign Application Priority Data
May 29, 2009 (DE) 10 2009 023 309.1

(51) Int. Cl. G02F 1/15; C07D 401/14; C07D 401/10; C09K 9/02

(52) U.S. Cl. 359/265; 252/583; 544/333; 546/101

(57) ABSTRACT

The service life of EC components can be substantially increased by eliminating the need for conducting salt additives, which can decompose the formulation by adverse reactions. At the same time, salt-like structures are formed by the active electrochromic components themselves.

### CROSS REFERENCE TO RELATED APPLICATIONS

This application is based on and hereby claims priority to International Application No. PCT/EP2010/055146 filed on Apr. 20, 2010 and German Application No. 10 2009 023 309.1 filed on May 29, 2009, the contents of which are hereby incorporated by reference.

### BACKGROUND

The invention relates to electrochromic materials, formulations thereof and the use thereof.

Electrochromic displays based on organic materials usually comprise an active electrochromic layer which, in the case of a display, is situated between electrodes arranged perpendicularly to one another. Essential components of the active layer are a redox system and an electrochromic dye. On application of an electric potential, the concentration ratio of the redox partners to one another in the material becomes shifted. During this reaction, protons and/or ions are released or bound in the material, and this affects the pH value. When an electric potential is applied to the material, the displacement of the equilibrium of the redox partners at the two electrodes takes place in the opposite direction. This has the effect that, for example, the pH value rises at one of the electrodes, whilst falling at the other electrode. Using a pH indicator dye, the change of the pH value is converted to a color change of the material and the application of the potential becomes visible.

WO 02/075441A2 and WO 02/075442 A1 disclose that a paste-like formulation that represents the electrochromic system is arranged between the electrodes. The composition of this electrochromic system comprises, as essential components, a polymer as a solid electrolyte, a conductive salt, a redox system, TiO₂ as a white pigment, a solvent and a dye. The latter is usually a pH indicator.

A further principle for realizing electrochromic displays lies therein that the color change is not brought about by the change in the pH value in the display, but by using the redox processes that occur anyway in order to generate contrast-rich color changes through the formation of reductive and/or oxidative states in suitable materials. Above all, the viologens and the polythiophenes have become known as classes of materials useful in this context. Examples in the literature are to be found in M. O. M. Edwards, Appl. Phys. Lett. 2005, 86(7) and Helmut W. Heuer, Rolf Wehrmann, Stephan Kirchmeyer Adv. Funct. Mater. 2002, 89.

As a rule, electrochromically active formulations are sought which change color rapidly on application of a potential and which, in the voltage-free state, rapidly return to the base state. This behavior is desirable for displays. For state displays such as ON/OFF displays, it is desirable to have formulations which, on reaching the respective switched state, remain for as long as possible, or permanently in the state.

Electrochromic systems of this type are known, for example, electrochromic mirrors or anchoring viologens on titanium crystals a) L. Walder, M. Möller, patent EP 1 271 227, 2003 b) M. Möller, S. Asaftei, D. Corr, M. Ryan, L. Walder, Adv. Mater. 2004, 16, 1558. However, these systems are technologically demanding, so that simple displays are too expensive.

From the electrochromic systems for which patent applications have already been made by the applicant, irreversibly switching electrochromic components known from DE 10 2006 015 056 and bistable electrochromic components known from DE 10 2006 045 307 can be made with little effort.

However, all the known electrochromic formulations suffer from the disadvantage that the compounds, which are frequently present in an ionogenic form, also contain simple counterions in the form, for example, of halide or alkali ions.

In the related art, the electrochromic species and the corresponding electrochromic counterparts are present alongside one another in the formulation and the presence of at least one conductive salt which represents an ion pool in the circuit of the electrochromic component, which, when the oxidation state of the active materials is altered, is partially used up and/or refilled. By increasing the voltage at geometrically defined regions of the graphics to be imaged (e.g. acute-angled image details) or the depiction of spikes on the electrode surfaces (typical for ITO electrodes) electrochemical conversion of the conductive salt additives can also occur, leading to severe service-life problems in the electrochromic components.

### SUMMARY

It is one possible object to increase the service life of electrochromic organic components.

The inventors propose a formulation having at least one electrochromically active compound, wherein the cationic and anionic species are ionogenic and are present in a compound, that is, salt-like and are mutually responsible for the charge exchange in the molecule, so that the use of simple counterions is unnecessary.

According to an advantageous embodiment, the formulation also comprises a white pigment for view blocking. Titanium oxide is preferably, though not exclusively, used as the white pigment.

According to an advantageous embodiment, the formulation also has a dispersing agent and/or a solvent, so that the formulation, the electrochromic compound and the white pigment in the formulation can be processed, for example, using a doctor blade method.

Electrochromically active materials have been successfully developed, the formulation of which is reduced, with regard to the essential active electrochromic components, such as anionic and cationic electrochromic systems, to only one organic compound. It is also possible for the compound to be included in a formulation which can be processed, in simple industrial processes and/or with minimum material usage, into displays.

The electrochromically active compound therefore contains oxidizing and reducing agents (that is, a redox pair) in an organic salt-type compound.

For this reason, with the inventors' proposals, as distinct from the related art, ions that are not needed in the redox process in the EC component are removed. This increases the simplicity of the EC component in the production thereof and therefore increases the service life thereof.

The subject matter of the proposal is therefore a redox-active compound which has the following general structure:

ₙ₊ₙ₋ [symbol]
(in equilibrium with)⁽ⁿ⁻¹⁾⁺⁽ⁿ⁻¹⁾⁻

Suitable cations that can act as oxidizing agents are loaded with electrodes at the cathode and suitable anions that can act as reducing agents have electrons removed at the anode. In this process, with the electrochromic component connected to a direct current source, the redox properties of the ions are reversed, so that, after the connection, the oxidizing agent is converted to a reducing agent and the reducing agent is converted to an oxidizing agent. The system relaxes when current-free (automatically) into the starting condition. With the change in the oxidization state of the anions and cations, the absorption of these ions can change strongly in the visible spectral region, depending on the structure thereof, or remain almost unchanged. This results in various application possibilities.

The change of polarity leads to the state brought about by the change in absorption being reversibly re-created at the electrodes.

It should be noted that the reduction potential of the oxidizing agent and the oxidation potential of the reducing agent have a difference (e.g. approximately 0.5V) such that, the agents cannot be connected into circuit without applying a potential. They would then change roles through internal electron transfer and would therefore not be suitable for use in EC components. In that event, the current-free colored materials could only be switched back into the colorless or white condition in an electric field, whereas without any power supplied, the system would revert again to the colored state.

A variety of systems can therefore be designed. Examples are the combinations:

### Cathodic EC Formulation:

ⁿ⁺ⁿ⁻are present in a colorless state, but in the switched-on state, that is, with⁽ⁿ⁻¹⁾⁺⁽ⁿ⁻¹⁾⁻, only the reducing agent is colored, so that the color thereof is visible.

### Anodic EC Formulation:

ⁿ⁺ⁿ⁻are present in a colorless state, but in the switched-on state, that is, with⁽ⁿ⁻¹⁾⁻, only the oxidizing agent is colored, so that the color thereof is visible.

### 2-Color Display:

ⁿ⁺ⁿ⁻are present in a colorless state, but in the switched-on state, that is, with⁽ⁿ⁻¹⁾⁺⁽ⁿ⁻¹⁾⁻, both the reducing agent and the oxidizing agent are colored, so that both colors can be made visible.

The case of a 2-color display is particularly interesting for a type of “traffic light” application, since both colored components are precipitated together as one salt. The compound/formulation is actually colorless, although when switched on, the compound becomes green at the cathode and red at the anode. The observer only ever sees one side of the display, so that application thereof, for example, as a signal display for traffic lights is possible. Other applications can be foreseen in simple displays for signaling switching states, danger situations, the elapse of time, etc.

The number of oxidizing agents that can be used is not restricted further, since many compounds can be used with the proposed solution. Systems that are usable as cathodically switchable electrochromic color systems or “oxidizing agents” are all systems which can absorb electrons at the cathode, e.g. 4,4′-bipyridine and all substituted forms thereof. Examples of cathodic electrochromic substances are, in particular, all the substances which are transformed by uptake of an electron at the cathode to a reduced species, with a change in the color appearance thereof:

Two typical representatives of such compounds, each with one example will now be given; all N,N′-disubstituted salt-like derivatives of bipyridines, such as poly-dodecylene-4,4″-bipyridinium-dibromide and all N,N″-disubstituted salt-like derivatives of 2,5-di(pyridin-4-yl)pyrimidine, such as N,N′-diheptyl-2,5-pyrimidinylen-di-4-pyridinium-dibromide.

The number of the reducing agents that are usable will not be further restricted here, since many compounds can be used with the present suggested solution.

Anodically switchable electrochromic “reducing agent” color systems that are usable are, for example, systems with the structure:

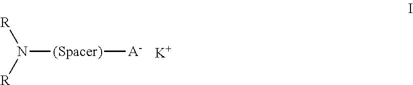

wherein “Spacer” stands for an alkylene group with up to 18 carbon atoms, preferably from 2 to 6 carbon atoms and particularly preferably with 4 carbon atoms,
R is selected from the group comprising alkyl and/or hydroxyalkyl groups with from 2 to 10 carbon atoms, phenyl-, p-tolyl-, m-tolyl-, and/or mesityl groups, groups suitable for the formation of dimers and polymers, that is groups which have a free valency, and suitable groups for bridging the groups R, so that the two groups R form a bidentate bond to the nitrogen, particularly—alkyl-groups with up to 10 carbon atoms in the chain, preferably from 2 to 6 carbon atoms,
A⁻ is an anion, for example, —SO₃⁻, —COO⁻, —O⁺,
and
K⁺ is an oxidizing agent as described above.

The “n” superscript in the formulae is the charge state of the cation or anion and is a digit ≧1, and given different charge states, the charge deficit is equalized through the stoichiometry of the relevant counterion. Particularly suitable, however, are cations and/or anions the charge state of which is at least 2 in the basic state, since in such cases, the reduced or oxidized species still have a charge state of at least one elemental charge with the potential applied and can therefore react particularly quickly to electric field changes, which is expressed in an increased switching speed. Single-charged ions are converted to neutral radical species, which are therefore only able to react much more slowly to electric field changes.

### DETAILED DESCRIPTION OF THE PREFERRED EMBODIMENT

Some representatives of the reducing agents and oxidizing agents will now be described in the form of preferred exemplary embodiments.

### Exemplary Embodiments

### A) Synthesis of Redox-Active Electrochromic Materials

### Example of an Electrochromic Device a)

preparation of the dicationic salt (bispyridylpyrimidine derivative) as per application 2006E24392.

Bromide: m.p.: 230-235° C., MS (ESI) cations m/2e=216.4

preparation of lithium-N-ethyl-N-mesityl butanesulfonate

1) mesitylacetamide (from mesitylamine and acetic anhydride)
2) N-ethyl-N-mesitylamine (reduction by lithium aluminum hydride) n_{D}²⁰=1.519
3) lithium-N-ethyl-N-mesityl butanesulfonate

Under extremely inert conditions (where possible, in a glove box) phenyllithium (slight excess) was added to N-ethyl-N-mesitylamine in absolute THF at −50° C. Following thawing, butane sultone was added.

After a short time, the product precipitated out in the form of an amorphous white salt (m.p. 295-300° C.), was drawn off and washed with ether.

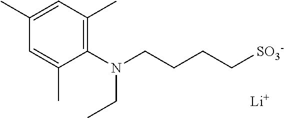

preparation of the redox-active electrochromic material:

The dicationic bispyridylpyrimidine derivative (see DE 10 2006 061 999.4) and the amine-substituted lithiumsulfonate were each individually dissolved in stoichiometric ratio to one another in methanol/water. After mixing of the solutions, a white salt-like product with the structure given below crystallized out, m.p.: 150-155° C. The product was drawn off and washed with ether. MS (ESI): Dication: m/2e=216.4 Anion: m/e=298.1

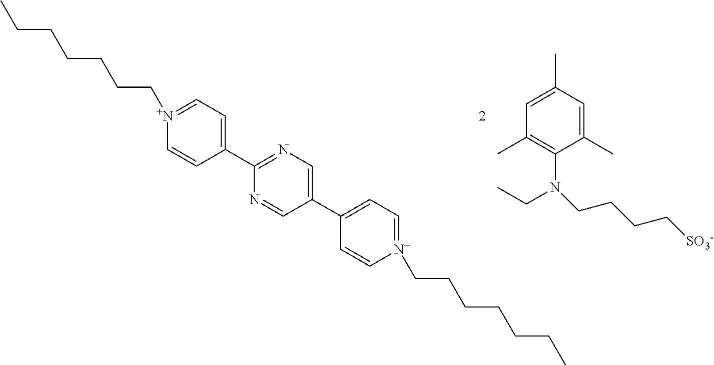

### Example of an Electrochromic Device b)

preparation of the dicationic species (substituted p-phenylene-bis-pyridinium salt) as per our application 2006E24129)

Tetrafluoroborate: m.p.: >310° C. MS (ESI) cations m/2e=345.5

preparation of the dianionic species (substituted 4,4″-bis-1-naphthylamine derivative) as per 2006E24471

Triethylammonium salt: m.p.: 160-165° C.

preparation of the redox-active electrochromic material:

The dicationic salt and the dianionic salt according to the aforementioned application were each individually dissolved in stoichiometric ratio to one another in methanol/water. After mixing of the solutions, a white salt-like product with the structure given below crystallized out, m.p.: 260-262° C. The product was drawn off and washed with ether. MS (ESI): Dication: m/2e=345.5 dianion: m/2e=317.9

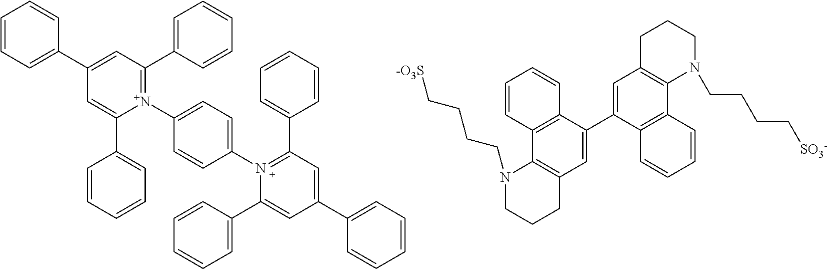

### Example of an Electrochromic Device c)

preparation of the dicationic salt (bispyridylpyrimidine derivative) as per application 2006E24392.

Bromide: m.p.: 230-5° C., MS (ESI) m/2e=216.4

preparation of the dianionic species (substituted 4,4″-bis-1-naphthylamine derivative) as per 2006E24471

Triethylammonium salt: m.p.: 160-165° C.

preparation of the redox-active electrochromic material:

the dicationic and the dianionic salt as per the aforementioned applications were each individually dissolved in stoichiometric ratio to one another in methanol/water. After mixing of the solutions, a white salt-like product with the structure given below crystallized out, m.p.: 180-185° C. The product was drawn off and washed with ether. MS (ESI): Dication: m/2e=216.4 Dianion: m/2e=317.9

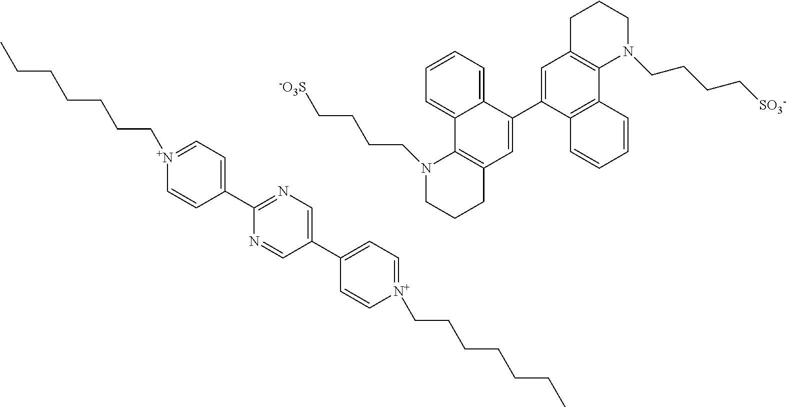

### B) Example for the production of the electrochromic pastes

1 part redox-active electrochromic material was mixed with 6 parts titanium dioxide and 2.2 parts diethylene glycol in a SpeedMixer for 5 min at 2000 rpm.

### C) Procedure for producing an electrochromic device

The electrochromic paste is applied with an application method (preferably with a doctor blade or by printing) between two electrodes, preferably ITO-coated PET films or glass plates, in a thickness in the range from 50 μm to 120 μm, particularly 70 μm, wherein the substrate coated with electrochromic paste is previously provided with an adhesive frame of the stated thickness in order to encapsulate the electrochromic device.

On application of a direct current potential to the electrodes of the device at the level of the oxidation potential of the respective anionic species (approximately 1V-2V), the device colors, depending on the redox-active electrochromic material used and on the poling applied. The coloration follows the pole-change. After switching off the voltage, the colored basic state, which is white, as given by the titanium dioxide is restored by reverse diffusion and charge equalization (usually in a few seconds).

With the inventors' proposals, it is possible significantly to increase the service life of the electrochromic components, since it is possible to dispense with the use of conductive salt additives that are capable of causing the decomposition of the formulation by side-reactions. Salt-like structures are formed by the electrochromically active components themselves.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1-7. (canceled)

8. A formulation comprising:
at least one electrochromically active compound, wherein salt-like cationic and anionic species are ionogenic and are present in the compound, the cationic and anionic species being mutually responsible for charge exchange in the formulation, so that simple counterions are unnecessary.

9. The formulation as claimed in claim 8, wherein the cationic species comprise an N,N′-disubstituted salt-like derivative of bipyridines.

10. The formulation as claimed in claim 8, wherein the anionic species is a molecule having the following structure: 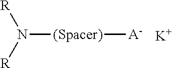
wherein “Spacer” stands for an alkylene group with up to 18 carbon atoms,
R is selected from the group consisting of alkyl and hydroxyalkyl groups with from 2 to 10 carbon atoms, phenyl-, p-tolyl-, m-tolyl-, and mesityl groups,
the two R groups are suitable for forming dimers and polymers, and have a free valency, for bridging the two groups R, so that the two groups R form a bidentate bond to the nitrogen,
A⁻ is an anion, and
K⁺ is an oxidizing agent.

11. The formulation as claimed in claim 9, wherein the anionic species is a molecule having the following structure: 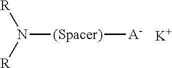
wherein “Spacer” stands for an alkylene group with 2 to 6 carbon atoms,
R is selected from the group consisting of alkyl and hydroxyalkyl groups with from 2 to 10 carbon atoms, phenyl-, p-tolyl-, m-tolyl-, and mesityl groups,
the two R groups are suitable for forming dimers and polymers, and have a free valency, for bridging the two groups R, so that the two groups R form a bidentate bond to the nitrogen,
A⁻ is an —SO₃⁻, —COO⁻, or —O⁻ anion, and
K⁺ is an oxidizing agent.

12. The formulation as claimed in claim 8, further comprising a white pigment.

13. The formulation as claimed in claim 8, further comprising a dispersing agent.

14. The formulation as claimed in claim 8, wherein the at least one electrochromically active compound is represented by the following formulae:

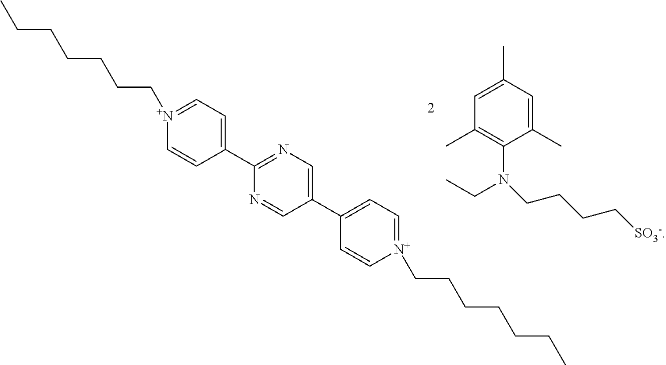

15. The formulation as claimed in claim 8, wherein the at least one electrochromically active compound is represented by the following formulae:

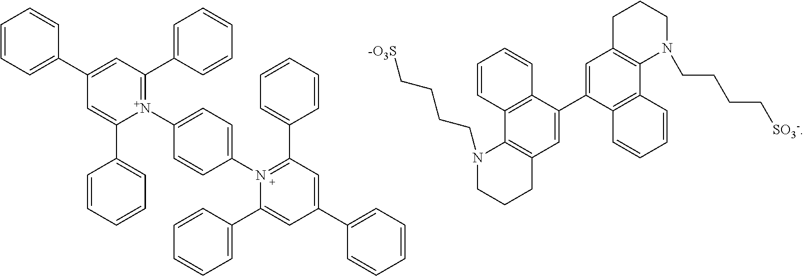

16. The formulation as claimed in claim 8, wherein the at least one electrochromically active compound is represented by the following formulae:

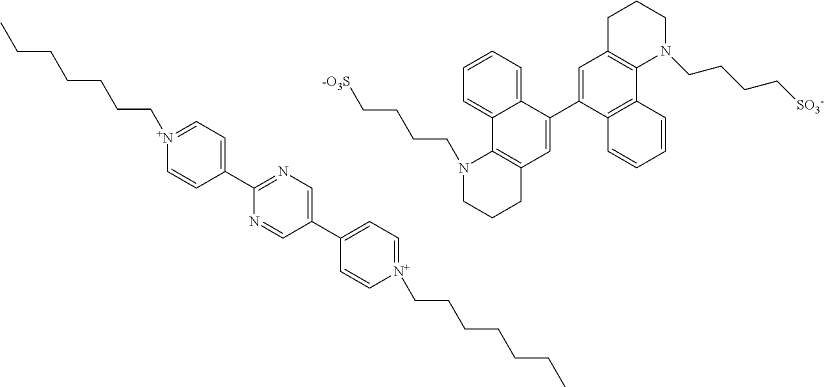

17. A method for producing a formulation, comprising:
providing at electrochromically active compound, wherein salt-like cationic and anionic species are ionogenic and are present in the compound;
mixing the electrochromically active compound into a dispersing agent and a white pigment, with a speed mixer, so that a paste formulation is produced having a viscosity such that the formulation can be processed using a doctor blade technique,
wherein the cationic and anionic species are mutually responsible for charge exchange in the formulation so that simple counterions are unnecessary.

18. An electrochromic organic component comprising:
at least first and second electrodes; and
an electrochromic formulation sandwiched between the first and second electrodes, the electrochromic formulation comprising at least one electrochromically active compound, wherein salt-like cationic and anionic species are ionogenic and are present in the compound, the cationic and anionic species being mutually responsible for charge exchange in the formulation, so that simple counterions are unnecessary.
